(19) United States
(12) Patent Application Publication
(10) Pub. No.: US 2015/0258117 A1
(43) Pub. Date: Sep. 17, 2015

(54) LOW-DOSE ESTRADIOL CREAM

(71) Applicant: Warner Chilcott Company, LLC, Fajardo, PR (US)

(72) Inventors: Tina M. deVries, Long Valley, NJ (US); Herman Ellman, Boonton Township, NJ (US)

(21) Appl. No.: 14/645,512

(22) Filed: Mar. 12, 2015

Related U.S. Application Data
(60) Provisional application No. 61/952,045, filed on Mar. 12, 2014.

(51) Int. Cl. A61K 31/565; A61K 9/107; A61K 9/00

(52) U.S. Cl. CPC A61K 31/565; A61K 9/0034; A61K 9/107

(57) ABSTRACT

A low-dose estradiol cream for vaginal administration that is safe and clinically effective to treat symptoms of menopause and a kit comprising the low-dose estradiol cream and an applicator are provided. A method of treatment of the symptoms of vulvar and/or vaginal atrophy associated with menopause, that includes the step of vaginally administering the low-dose estradiol cream to a woman in need thereof is also provided.

### CROSS-REFERENCE TO RELATED APPLICATION

This application claims the benefit of U.S. Provisional Patent Application No. 61/952,045, filed Mar. 12, 2014, the entire disclosure of which is incorporated by reference herein.

### BACKGROUND OF THE INVENTION

1. Field of the Invention

This invention relates to a low-dose estradiol cream for vaginal administration that is safe and clinically effective to treat symptoms of menopause and a kit comprising the low-dose estradiol cream and an applicator. This invention is also related to a method of treatment of the symptoms of vulvar and/or vaginal atrophy associated with menopause, such as vaginal dryness and dyspareunia, that includes the step of administering the low-dose estradiol cream to a woman in need thereof.

2. Description of Related Art

Vaginal dryness and dyspareunia are symptoms of vulvar and/or vaginal atrophy, and are commonly associated with menopause. Conventional treatments include semi-solid estrogen vaginal preparations, which comprise a two-phase emulsified system. Such conventional estrogen vaginal preparations have a significant hydrophobic oil or wax component, present either as the external or, more typically, the internal phase of a two-phase emulsified system, where water constitutes the other phase. Cream-based estradiol formulations tend to have relatively high estradiol concentrations, such as found in Estrace® or Premarin®. Other estradiol formulations for vaginal treatment are known in the art, such as Vagifem® (estradiol vaginal tablets). However, vaginal tablets lack the comfort of vaginal creams.

U.S. Application Publication No. 2006/0292223 relates to a pharmaceutical gel composition for topical administration comprising (a) at least one pharmacologically active agent, such as estrogen, in an amount of about 0.00001% to about 10% by weight of the composition; (b) at least one hydrogen-bonding gelation polymer; and (c) at least one gelation promoter in an amount effective to at least partially solubilize the pharmacologically active agent and to gel the polymer, wherein at least a portion of the pharmacologically active agent is dissolved in the composition at 15° C.

U.S. Application Publication No. 2007/0004694 relates to a pharmaceutical gel composition containing estrogen for vaginal administration comprising (a) at least one estrogen in an amount of about 0.00001% to about 2% by weight of the composition; (b) at least one hydrogen-bonding gelation polymer; and (c) at least one gelation promoter in an amount effective to substantially solubilize the estrogen and to gel the polymer, wherein at least a portion of the estrogen is dissolved in the composition at 15° C.

U.S. Application Publication No. 2007/0004693 relates to a pharmaceutical gel composition containing estrogen for vaginal administration comprising (a) at least one estrogen in an amount of about 0.00028% to about 1% by weight of the composition; (b) at least one gelation polymer; and (c) at least one aqueous solvent, wherein a portion of the estrogen in the composition is in suspension at 15° C.

The foregoing gel compositions formulations have not been shown so far to be safe and clinically effective.

None of the references referred to above discloses that a relatively small dose of estradiol cream, i.e., a 0.5 gram dose, containing about 0.003% or less of estradiol was effective for treating symptoms of vulvar and/or vaginal atrophy associated with menopause.

A safe and clinically effective low-dose estradiol cream for vaginal administration is important for providing effective relief of certain symptoms of menopause with a relatively low dose of estrogen, which simultaneously minimizes systemic exposure to estradiol in a convenient and comfortable dosage form.

### SUMMARY OF THE INVENTION

The present invention is directed to a low-dose estradiol cream for vaginal administration that is safe and clinically effective to treat the symptoms of menopause. Each dose of the estradiol cream is about 0.5 gram of the estradiol cream containing about 0.015 mg or less of estradiol, its salts, esters, hydrates, prodrugs or derivatives thereof. The estradiol cream is further comprised of a pharmaceutically acceptable cream vehicle, which preferably is an oil-in-water emulsion. Preferably, creams of the invention comprise relatively low amounts of oil, e.g., about 5% wt./wt. or less, about 4% wt./wt. or less, about 3% wt./wt. or less, about 2% wt./wt. or less, and most preferably about 1% wt./wt. or less.

Another embodiment of the present invention is directed to a method of treatment of symptoms of vulvar and/or vaginal atrophy associated with menopause comprising the step of vaginally administering to a woman in need thereof a low-dose estradiol cream that is safe and clinically effective to treat the symptoms of menopause. Each administration is a dose in an amount of about 0.5 g of the estradiol cream containing about 0.015 mg or less of estradiol, its salts, esters, hydrates, prodrugs or derivatives thereof. The estradiol cream is further comprised of a pharmaceutically acceptable cream vehicle, which preferably is an oil-in-water emulsion.

Yet another embodiment of the present invention is directed to a kit comprising a low-dose estradiol cream for vaginal administration that is safe and clinically effective to treat the symptoms of menopause and an applicator, wherein each dose of the estradiol cream is about 0.5 gram of the estradiol cream containing about 0.015 mg or less of estradiol, its salts, esters, hydrates, prodrugs or derivatives thereof.

### DETAILED DESCRIPTION OF THE INVENTION

An embodiment of the present invention is directed to a low-dose estradiol cream for vaginal administration that is safe and clinically effective to treat the symptoms of menopause. Each dose of the estradiol cream is about 0.5 grams of the estradiol cream containing about 0.015 mg or less of estradiol, its salts, esters, hydrates, prodrugs or derivatives thereof. The estradiol cream is further comprised of a pharmaceutically acceptable cream vehicle, which preferably is an oil-in-water emulsion.

While it is desirable in the present invention to deliver a dose of 0.5 grams, a person of ordinary skill will recognize that delivery of such a dose will inherently have a slight amount of variability that will be dependent on the applicator being used and the technique of the patient. Taking into account such variability, it is expected that each dose of estradiol cream may range in weight from 0.427 g to 0.564 g of the estradiol cream.

In another embodiment of the present invention, each dose of the estradiol cream is about 0.5 gram of the estradiol cream containing a range of about 0.015 mg to about 0.009 mg, more preferably about 0.015 mg to about 0.011 mg, even more preferably about 0.015 mg to about 0.013 mg of estradiol, its salts, esters, hydrates, prodrugs or derivatives thereof. Each about 0.5 gram dose of estradiol cream most preferably contains about 0.015 mg of estradiol. In other embodiments of the present invention, the estradiol cream may contain 0.014 mg, 0.013 mg, 0.012 mg, 0.011 mg, 0.010 mg or 0.009 mg of estradiol, its salts, esters, hydrates, prodrugs or derivatives thereof in about 0.5 grams of estradiol cream.

In an embodiment, each dose of the estradiol cream is about 0.5 gram of the estradiol cream containing 0.015 mg of estradiol, its salts, esters, hydrates, prodrugs or derivatives thereof, and a pharmaceutically acceptable cream vehicle. Such a dose may also be referred to as a 0.003% estradiol cream.

In a preferred embodiment, estradiol is 17β-estradiol.

As used herein, “pharmaceutically acceptable” means the component is appropriate for application to the vaginal environment.

As used herein, “safe and clinically effective” means a drug formulation that meets the standards for safety and effectiveness required by the United States Food and Drug Administration.

The term “salt”, as used herein, refers to a salt that is pharmaceutically acceptable and that possesses the desired pharmacological activity of the parent compound. Nonlimiting examples of salts include (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth metal ion, or an aluminum ion; or coordinates with an organic base such as ethanolamine, diethanolamine, triethanolamine, tromethamine, N-methylglucamine, and the like. Nonlimiting examples of the salts of 17β-estradiol include, 17β-estradiol hydrochloride salt, β-estradiol 17-(β-D-glucuronide) sodium salt and β-estradiol 3-(β-D-glucuronide) 17-sulfate dipotassium salt.

The term “ester”, as used herein, refers to an organic compound made by replacing the hydrogen of an acid by an alkyl, e.g., C₁ to C₆ alkyl, or other organic group. Various esters of estradiol are well known in the art. Nonlimiting examples of esters of estradiol, and in particular, of 17β-estradiol, include, lower alkanoate esters such as 17β-estradiol-3-formate, 17β-estradiol-3-acetate, 17β-estradiol-3-propionate and mixtures thereof; estradiol-3,17-diacetate, estradiol-3,17-valerate, estradiol-3-valerate, estradiol-17-valerate, estradiol 3-benzoate, estradiol cypionate, estradiol dipropionate, and estradiol enantate. Most preferably, the 17β-estradiol-3-lower alkanoate is 17β-estradiol-estradiol-3-acetate.

The term “hydrate”, as used herein, refers to a compound formed by the addition of water. The hydrates may be obtained by any known method in the art by dissolving the compounds in water and recrystallizing them to incorporate water into the crystalline structure. Nonlimiting examples of hydrates include hemihydrate, monohydrate, dehydrate, trihydrate, and pentahydrate. In a preferred embodiment, the hydrate of 17β-estradiol is 17β-estradiol hemihydrate.

The term “prodrug”, as used herein, refers to an inactive precursor of a drug, converted into its active form in the body by normal metabolic processes. Various forms of prodrugs are well known in the art. Nonlimiting examples of prodrugs of 17β-estradiol include, the prodrug described in U.S. Pat. No. 7,067,505.

The estradiol cream of the present invention comprises a pharmaceutically acceptable cream vehicle.

The pharmaceutically acceptable cream vehicle is preferably an oil-in-water emulsion. In an embodiment of the present invention, the oil content is less than about 5% wt./wt. of the estradiol cream. In another embodiment of the present invention, the oil content is less than about 1% wt./wt. of the estradiol cream.

In an embodiment of the present invention, the pharmaceutically acceptable cream vehicle comprises one or more solvents, a viscosity modifier, an emollient and a surfactant. The amounts of solvent(s), viscosity modifier, emollient and surfactant that may be used in the cream vehicle are understood by those skilled in the art.

In an embodiment of the present invention, any pharmaceutically acceptable solvent can be used. Non-limiting examples of suitable solvent include propylene glycol, polyethylene glycol, water, and combinations thereof. In a preferred embodiment, the solvent is a combination of propylene glycol, polyethylene glycol and water.

In an embodiment of the present invention, the polyethylene glycol has a molecular weight range of about 200 to about 2,000. In a preferred embodiment, the polyethylene glycol is PEG 200, PEG 300, PEG 400 or PEG 600. In an even more preferred embodiment, the polyethylene glycol is PEG 400.

The pharmaceutically acceptable cream vehicle may also comprise a viscosity modifier. Nonlimiting examples of the viscosity modifier include polyacrylic acids. Polyacrylic acid of the present invention may be homopolymers of acrylic acid, crosslinked with an allyl ether pentaerythritol, allyl ether of sucrose, allyl ether of propylene or divinyl glycol. Non-limiting examples of suitable polyacrylic acid include polycarbophil and carbomer copolymer type A (e.g., commercially available under the tradename Pemulen™ TR-2). In a preferred embodiment, the viscosity modifier is polycarbophil.

The pharmaceutically acceptable cream vehicle may also comprise an emollient. Nonlimiting examples of the emollient include pharmaceutically acceptable oils. Nonlimiting examples of the pharmaceutically acceptable oils include castor oil, medium chain triglycerides (MCTs), mineral oils, vegetable oils, oily fatty acids, oily fatty alcohols, esters of sorbitol, fatty acids, oily sucrose esters, and any combination thereof. In a preferred embodiment, the emollient is mineral oil.

The pharmaceutically acceptable cream vehicle may also comprise a surfactant. Nonlimiting examples of the surfactant include polysorbates and poloxamers. In a preferred embodiment, the surfactant is polysorbate 80.

In a preferred embodiment, the pharmaceutically acceptable cream vehicle may comprise propylene glycol, polyethylene glycol, polyacrylic acid, mineral oil, polysorbate and water.

In an embodiment of the present invention, the low-dose estradiol cream further comprises an effective amount of an antioxidant. Any GRAS (“generally recognized as safe” by the U.S. Food & Drug Administration) antioxidant is suitable for use in the present invention. Non-limiting examples of suitable antioxidants include tocopherol, butylated hydroxytoluene, butylated hydroxyanisole, dodecyl gallate, octyl gallate, propyl gallate, ascorbyl palmitate, sodium ascorbate, thymol and combinations thereof. The amount of antioxidant that may be used in the cream is understood by those skilled in the art.

Another embodiment of the present invention is a method of treatment of symptoms of vulvar and/or vaginal atrophy associated with menopause comprising the step of vaginally administering the low-dose estradiol cream of the invention to a woman in need thereof. The estradiol cream is safe and clinically effective to treat the symptoms of menopause. In an embodiment, each administration is a dose in an amount of about 0.5 g of the estradiol cream containing about 0.015 mg or less of estradiol, its salts, esters, hydrates, prodrugs or derivatives thereof. The estradiol cream is further comprised of a pharmaceutically acceptable cream vehicle, which preferably is an oil-in-water emulsion.

Nonlimiting examples of the symptoms of vulvar and/or vaginal atrophy associated with menopause include vaginal dryness, dyspareunia (painful intercourse), vaginal and/or vulvar irritation/itching, dysuria (painful urination), and vaginal bleeding associated with sexual activity.

The estradiol cream of the present invention may be vaginally administered using an applicator. The applicator is preferably metered to dispense each dose in an amount of about 0.5 g of the estradiol cream.

The estradiol cream of the invention may be administered as needed, e.g., daily, twice daily, weekly, twice weekly, thrice weekly, etc. The administration may also be varied while the patient is being treated. For example, in an embodiment of the present invention, the estradiol cream may be administered once daily for two weeks followed by twice weekly doses for the treatment of vaginal dryness. In a preferred embodiment, the total treatment duration is twelve weeks. Preferred treatment durations may also extend for a year or more.

In another embodiment of the present invention, the estradiol cream may be administered once daily for two weeks followed by thrice weekly doses for the treatment of dyspareunia. In a preferred embodiment, the total treatment duration is twelve weeks. Preferred treatment durations may also extend for a year or more.

Yet another embodiment of the present invention is a kit comprising a low-dose estradiol cream for vaginal administration and an applicator, wherein each dose of the estradiol cream is about 0.5 gram of the estradiol cream containing about 0.015 mg or less of estradiol, its salts, esters, hydrates, prodrugs or derivatives thereof. The estradiol cream may be vaginally administered using the applicator. The applicator is preferably metered to dispense each dose in an amount of about 0.5 g of the estradiol cream.

A specific embodiment of the invention will now be demonstrated by reference to the following examples. It should be understood that these examples are disclosed by way of illustrating the invention and should not be taken in any way to limit the scope of the present invention.

### Examples & Comparative Example

Safety and efficacy of various estradiol creams were evaluated in postmenopausal women and were compared with the safety and efficacy of vehicle (vaginal cream). The efficacy was determined using three co-primary endpoints: The subject's self-assessment of the severity of vaginal dryness or dyspareunia, the change from baseline in vaginal pH and vaginal cytology (superficial cells and parabasal cells) at the final week of treatment were compared with those at the beginning of the treatment. The safety was assessed by monitoring patients, including adverse events and vital signs.

Table 1 shows the composition of Comparative Example 1 whose safety and efficacy was studied. Each dose was about 1 gram of estradiol cream containing 0.017 mg of estradiol. Such a dose may also be referred to as a 0.0017% estradiol cream.

TABLE 1
Composition of Comparative Example 1
(Estradiol Vaginal Cream, 0.0017%)

| | | Amount/Dose |
|---|---|---|
| | | (mg/1.0 gram |
| Ingredients | Function | cream) |
| Estradiol | Active ingredient | 0.017 |
| Propylene glycol | Solvent | 140.0 |
| Polyethylene glycol | Solvent | 741.5 |
| 400 | | |
| Polycarbophil | Viscosity modifier | 17.5 |
| Purified water | Solvent | 90.0 |
| Mineral oil | Emollient | 10.0 |
| Polysorbate 80 | Surfactant | 1.00 |

The cream was packaged in aluminum tubes (sealed closure) with a polypropylene cap.

The safety and efficacy of Comparative Example 1 (0.0017%) estradiol vaginal cream was compared to vehicle (vaginal cream) in postmenopausal women for the relief of vaginal dryness due to vulvovaginal atrophy with a dosing regimen of daily vaginal administration of about 1 gram of estradiol cream containing 0.017 mg of estradiol for 2 weeks followed by either twice or thrice weekly doses for 10 weeks for total treatment duration of 12 weeks.

The treatment with Comparative Example 1 estradiol vaginal cream 2 or 3 times per week was safe and well-tolerated in postmenopausal subjects.

The difference between estradiol vaginal cream (0.0017%) and vehicle (thrice weekly) in the change from baseline with respect to the subject's self-assessment of the severity of vaginal dryness was not statistically significant at week 12/final visit. The difference between 0.0017% estradiol vaginal cream and vehicle thrice weekly was statistically significant for the change from baseline in vaginal pH and vaginal cytology (superficial cells and parabasal cells). The difference between 0.0017% estradiol vaginal cream and vehicle twice weekly was also statistically significant for the change from baseline in vaginal pH and vaginal cytology (superficial cells and parabasal cells).

However, the change in the subject's self-assessment of the severity of vaginal dryness for 0.0017% estradiol vaginal cream thrice weekly was not statistically significant, and therefore it could not be concluded that there is a difference between the efficacy of 0.0017% estradiol vaginal cream and vehicle for the relief of vaginal dryness. While the results for twice weekly treatment of vaginal dryness appear positive, the failure of the thrice weekly treatment prevents a conclusion of efficacy for the formulation of Comparative Example 1.

It was also observed that 0.0017% estradiol vaginal cream did not demonstrate clinical effectiveness regarding dyspareunia.

The inventors of the present invention developed a new low-dose formulation of the estradiol cream, Example 1. Each dose of the estradiol cream is about 0.5 gram of the estradiol cream containing 0.015 mg of estradiol. Such a dose may also be referred to as a 0.003% estradiol cream. The composition of Example 1 is shown in Table 2.

TABLE 2
Composition of Example 1 (Estradiol Vaginal Cream, 0.003%)

| | | Amount/Dose |
|---|---|---|
| | | (mg/0.5 gram |
| Ingredients | Function | cream) |
| Estradiol | Active ingredient | 0.015 |
| Propylene glycol | Solvent | 70.00 |
| Polyethylene glycol | Solvent | 370.7 |
| 400 | | |
| Polycarbophil | Viscosity modifier | 8.750 |
| Purified water | Solvent | 45.00 |
| Mineral oil | Emollient | 5.000 |
| Polysorbate 80 | Surfactant | 0.500 |

The cream was packaged in aluminum tubes (sealed closure) with a polypropylene cap.

The safety and efficacy of the Example 1 (0.003%) estradiol vaginal cream was compared to vehicle (vaginal cream) in postmenopausal women for the relief of vaginal dryness and/or dyspareunia due to vulvovaginal atrophy with a dosing regimen of daily vaginal administration of daily vaginal administration of about 0.5 gram of estradiol cream containing 0.015 mg of estradiol for 2 weeks followed by either twice or thrice weekly doses for 10 weeks for total treatment duration of 12 weeks.

The treatment with Example 1 estradiol vaginal cream 2 or 3 times per week was safe and well-tolerated in postmenopausal subjects.

Moreover, unlike Comparative Example 1, a statistically significant difference in the change from baseline to final assessment (the last available post-baseline assessment) was observed between 0.003% estradiol cream and vehicle for all three co-primary endpoints, namely, subject self-assessment for dryness (twice weekly dosing) and dyspareunia (thrice weekly dosing), vaginal cytology and vaginal pH. Based on the success of the formulation of Example 1, it can be concluded that this formulation is both safe and clinically effective.

Example 1 had a lower dose of 0.015 mg estradiol as compared to the dose of 0.017 mg estradiol of Comparative Example 1. Yet, only Example 1 was concluded to be efficacious when compared to the vehicle. The efficacy when using a smaller volume of cream, which contains less estradiol (Example 1) as compared to the a larger volume of the analogous cream containing more estradiol (Comparative Example 1) was the opposite of what one might expect and was surprising in that the 0.015 mg cream was efficacious at all given the failure of Comparative Example 1.

While the invention has been described above with reference to specific embodiments thereof, it is apparent that many changes, modifications, and variations can be made without departing from the inventive concept disclosed herein. Accordingly, it is intended to embrace all such changes, modifications, and variations that fall within the spirit and broad scope of the appended claims. All patent applications, patents, and other publications cited herein are incorporated by reference in their entirety.

What is claimed is:

1. A low-dose estradiol cream for vaginal administration,
wherein each dose of the estradiol cream is about 0.5 gram of the estradiol cream containing about 0.015 mg or less of estradiol, its salts, esters, hydrates, prodrugs or derivatives thereof and a pharmaceutically acceptable cream vehicle; and
wherein the estradiol cream is safe and clinically effective to treat symptoms of menopause.

2. The low-dose estradiol cream of claim 1, wherein each dose of the estradiol cream is about 0.5 gram of the estradiol cream containing about 0.015 mg of estradiol, its salts, esters, hydrates, prodrugs or derivatives thereof.

3. The low-dose estradiol cream of claim 1, wherein the pharmaceutically acceptable cream vehicle is an oil-in-water emulsion.

4. The low-dose estradiol cream of claim 3, wherein the oil content is less than about 5% wt./wt. of the estradiol cream.

5. The low-dose estradiol cream of claim 3, wherein the oil content is less than about 1% wt./wt. of the estradiol cream.

6. The low-dose estradiol cream of claim 3, wherein the pharmaceutically acceptable cream vehicle comprises one or more solvents, a viscosity modifier, an emollient and a surfactant.

7. The low-dose estradiol cream of claim 6, wherein the solvent is selected from the group consisting of propylene glycol, polyethylene glycol, water and combinations thereof.

8. The low-dose estradiol cream of claim 6, wherein the viscosity modifier is a polyacrylic acid.

9. The low-dose estradiol cream of claim 6, wherein the emollient is mineral oil.

10. The low-dose estradiol cream of claim 6, wherein the surfactant is polysorbate.

11. The low-dose estradiol cream of claim 1, wherein the pharmaceutically acceptable cream vehicle comprises propylene glycol, polyethylene glycol, polyacrylic acid, mineral oil, polysorbate and water.

12. The low-dose estradiol cream of claim 11, wherein the polyethylene glycol is PEG 400.

13. The low-dose estradiol cream of claim 11, wherein the polyacrylic acid is polycarbophil.

14. The low-dose estradiol cream of claim 1, wherein the low-dose estradiol cream further comprises an effective amount of an antioxidant.

15. A method of treatment of symptoms of vulvar and/or vaginal atrophy associated with menopause comprising the step of vaginally administering a low-dose estradiol cream to a woman in need thereof,
wherein each administration is a dose in an amount of about 0.5 g of the estradiol cream containing about 0.015 mg or less of estradiol, its salts, esters, hydrates, prodrugs or derivatives thereof and
a pharmaceutically acceptable cream vehicle; and
wherein the estradiol cream is safe and clinically effective to treat symptoms of menopause.

16. The method of treatment of claim 15, wherein each dose of the estradiol cream is about 0.5 gram of the estradiol cream containing about 0.015 mg of estradiol, its salts, esters, hydrates, prodrugs or derivatives thereof.

17. The method of treatment of claim 15, wherein the pharmaceutically acceptable cream vehicle is an oil-in-water emulsion.

18. The method of treatment of claim 17, wherein the oil content is less than about 5% wt./wt. of the estradiol cream.

19. The method of treatment of claim 17, wherein the oil content is less than about 1% wt./wt. of the estradiol cream.

20. The method of treatment of claim 17, wherein the pharmaceutically acceptable cream vehicle comprises one or more solvents, a viscosity modifier, an emollient and a surfactant.

21. The method of treatment of claim 15, wherein each dose of the estradiol cream is administered daily or twice daily.

22. The method of treatment of claim 15, wherein each dose of the estradiol cream is administered once weekly, twice weekly or thrice weekly.

23. The method of treatment of claim 15, wherein the estradiol cream is administered using an applicator.

24. The method of treatment of claim 23, wherein the applicator is metered to dispense each dose in an amount of about 0.5 grams of the estradiol cream.

25. The method of treatment of claim 15, wherein the symptoms of vulvar and/or vaginal atrophy associated with menopause include vaginal dryness, dyspareunia, vaginal and/or vulvar irritation/itching, dysuria, or vaginal bleeding associated with sexual activity.

26. The method of treatment of claim 15, wherein the estradiol cream is administered once daily for two weeks followed by twice weekly doses for the treatment of vaginal dryness.

27. The method of treatment of claim 15, wherein the estradiol cream is administered once daily for two weeks followed by thrice weekly doses for the treatment of dyspareunia.

28. A kit comprising a low-dose estradiol cream for vaginal administration and an applicator,
wherein each dose of the estradiol cream is about 0.5 gram of the estradiol cream containing about 0.015 mg or less of estradiol, its salts, esters, hydrates, prodrugs or derivatives thereof and
a pharmaceutically acceptable cream vehicle; and
wherein the estradiol cream is safe and clinically effective to treat symptoms of menopause.
